(19) United States
(12) Patent Application Publication
(10) Pub. No.: US 2024/0228460 A1
(43) Pub. Date: Jul. 11, 2024

(54) PROTEASE INHIBITORS AND METHODS OF MAKING SAME

(71) Applicant: Variational AI Inc., Vancouver (CA)

(72) Inventors: Jason ROLFE, Vancouver (CA); Mehran KHODABANDEH, Vancouver (CA); Zaccary ALPERSTEIN, Cambridge, MA (US); Seyed Ali SABERALI, Vancouver (CA); Hazem MSLATI, Montreal (CA); Peter GUZZO, Niskayuna, NY (US)

(21) Appl. No.: 18/390,559

(22) Filed: Dec. 20, 2023

Related U.S. Application Data
(60) Provisional application No. 63/476,516, filed on Dec. 21, 2022.

(51) Int. Cl. C07D 401/12; A61K 31/40; A61K 31/428; A61K 31/4365; A61K 31/437; A61K 31/4439; A61K 31/4725; A61K 45/06; A61P 31/14; C07D 207/34; C07D 417/12; C07D 471/04; C07D 495/04

(52) U.S. Cl. CPC C07D 401/12; A61K 31/40; A61K 31/428; A61K 31/4365; A61K 31/437; A61K 31/4439; A61K 31/4725; A61K 45/06; A61P 31/14; C07D 207/34; C07D 417/12; C07D 471/04; C07D 495/04

(57) ABSTRACT

Inhibitors of the SARS-CoV-2 M^{pro} protease and methods of their use in treating a coronavirus infection in a subject are provided. The inhibitors may have the general structure (I):

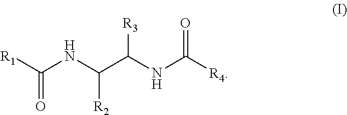

### CROSS-REFERENCE TO RELATED APPLICATIONS

This application claims priority to, and the benefit of, U.S. provisional patent application No. 63/476,516 filed 21 Dec. 2022, the entirety of which is incorporated by reference herein for all purposes.

### SEQUENCE LISTING

This application contains a Sequence Listing which has been filed electronically in XML format and is hereby incorporated by reference herein in its entirety. Such XML copy, created on 4 Dec. 2023, is called V273 0003 US.xml and is 3 kilobytes in size.

### TECHNICAL FIELD

Some embodiments relate to protease inhibitors and methods of their use. Some embodiments relate to inhibitors of coronavirus proteases and their methods to treat coronavirus. Some embodiments relate to inhibitors of the SARS-CoV-2 main protease (M^{pro}) and methods of their use, for example to treat COVID-19. Some embodiments relate to inhibitors of cathepsin L and methods of their use, for example to treat COVID-19.

### BACKGROUND

The COVID-19 pandemic illustrated the ease with which an unchecked pathogen can interfere with the normal activities of human society. A great deal of effort was expended to try to quickly identify and approve therapies and vaccines that could help to check the spread of the pandemic by treating or limiting the spread of the SARS-CoV-2 virus that causes COVID-19.

Despite much success in developing vaccines and antibody therapies against the SARS-CoV-2 virus that causes COVID-19, there still is no small molecule antiviral drug that is highly effective in treating patients suffering from the virus. Current front-line therapy includes antivirals such as remdesivir or Paxlovid™ (nirmatrelvir and ritonavir), which have been used with somewhat limited success and/or which may carry undesirable side effects.

The SARS-CoV-2 M^{pro} protease represents an attractive target for COVID-19 therapeutics. The protease is essential for the maturation and replication of the SARS-CoV-2 virus, and its protease substrate binding pocket is highly similar across multiple different coronaviruses. Thus, therapeutic agents targeting the M^{pro} protease may potentially be effective across multiple variants of the virus.

There is also some evidence that cathepsin L is implicated in entry of the SARS-CoV-2 virus into cells by activating the viral spike protein in the endosome or lysosome, and some studies have shown that inhibitors of cathepsin L can block or decrease the entry of SARS-CoV-2 into cells without being toxic to the mammalian host. Thus, inhibitors of cathepsin L, and in particular compounds that are active as inhibitors of both SARS-CoV-2 M^{pro} and cathepsin L, may have utility as therapeutic agents in the treatment of SARS-CoV-2.

There is further evidence that inhibitors of SARS-CoV-2 M^{pro} may also have inhibitory effects against M^{pro} from other coronavirus types that are known to infect humans, including for example other beta-coronaviruses including SARS-CoV-1, MERS-CoV, HCoV-OC43 and HCoV-HKU1, and alpha-coronaviruses including HCoV-229E and HCoV-NL63 (see e.g. Owen et al., 2021; Unoh et al, 2022).

There thus remains a need for small molecule therapeutics that can be used to treat patients suffering from infection with SARS-CoV-2 and other coronaviruses. There further remains a need for rapid drug discovery mechanisms that can be used to quickly identify potential therapeutic candidates and accelerate the selection of lead compounds for use as such therapeutics.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In some aspects, a compound having the following structure (I) or a pharmaceutically acceptable salt thereof is provided:

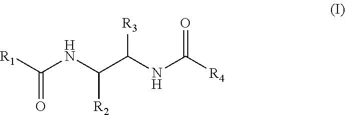

wherein:
R₁ is

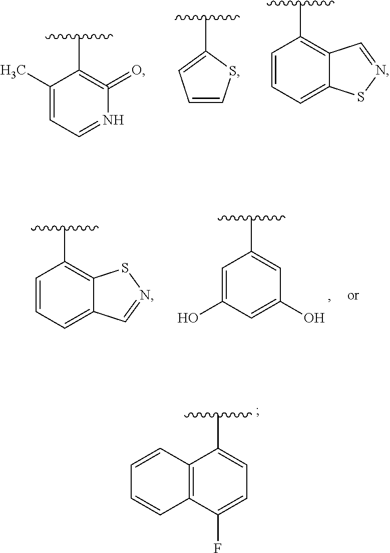

R₂ is H;
R₃ is

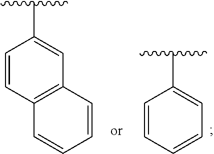 [SMILES: *c1ccc2ccccc2c1 ; *c1ccccc1]

and
R₄ is

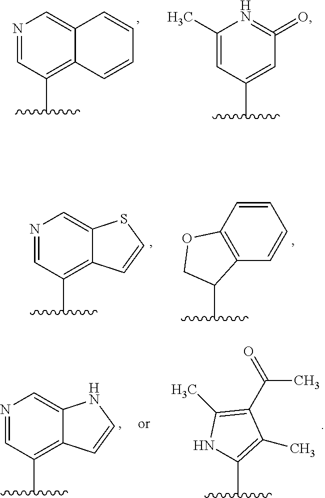

In some aspects, a pharmaceutical composition or an oral dosage form containing such a compound is provided. In some aspects, a method of inhibiting a main protease (M^{pro}) of a coronavirus is provided in which the M^{pro} is exposed to a compound, pharmaceutically acceptable salt, pharmaceutical composition or oral dosage form as described herein. In some aspects, the method further includes inhibiting cathepsin L. In some aspects, the compound, pharmaceutically acceptable salt, pharmaceutical composition or oral dosage form is administered to a mammalian subject. In some aspects, the coronavirus is a beta-coronavirus or an alpha-coronavirus, including for example the alpha-coronavirus being HCoV-229E or HCoV-NL63; the beta-coronavirus being SARS-CoV, MERS-CoV, HCoV-OC43, or HCoV-HKU1; or the SARS-CoV being SARS-CoV-1 or SARS-CoV-2.

In some aspects, the compound, pharmaceutical composition or oral dosage form is administered together with a second antiviral, an immune modulator, or a monoclonal antibody treatment. The second antiviral can be remdesivir, molnupiravir, ritonavir, nirmatrelvir or ritonavir/nirmatrelvir; the immune modulator can be baricitinib; or the monoclonal antibody treatment can be bebtelovimab, tixagevimab, cilgavimab, bamlanivimab, etesevimab, casirivimab, imdevimab, or sotrovimab. Kits containing the compound, pharmaceutical composition or oral dosage form can be provided together with instructions for carrying out the methods described herein. In some aspects, the compound can be used in a method of manufacturing a medicament for use in treatment of a coronavirus infection in a mammalian subject.

In addition to the exemplary aspects and embodiments described above, further aspects and embodiments will become apparent by study of the following detailed descriptions.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The inventors have now discovered novel compounds that are useful as inhibitors of SARS-CoV-2 M^{pro} and correspondingly are potentially useful as therapeutic agents for the treatment of infections caused by SARS-CoV-2. Further, since the substrate binding pocket of M^{pro} is highly conserved across different strains of coronavirus and other compounds that are active as inhibitors of SARS-CoV-2 M^{pro} have also been demonstrated to possess activity against the M^{pro} of other coronaviruses, such compounds are potentially useful in the treatment of multiple strains of coronavirus, including for example other beta-coronavirus strains including SARS-CoV-1, CoV MERS, MERS-CoV, HCoV-OC43, HCoV-HKU1, or the like and alpha-coronavirus strains including HCoV-229E, HCoV-NL63, or the like (see e.g. Owen et al., 2021; Unoh et al., 2022).

Discovery of the novel compounds of this invention was facilitated with Enki™ Generative AI, a powerful generative artificial intelligence platform developed by Variational AI Inc. (Vancouver, Canada). Enki™ Generative AI allows predictive generation of de novo molecules with properties optimized for pharmaceutical efficacy, safety and synthesizability.

As used herein, the terms “treat” and “treatment” encompass prophylactic or preventative treatment, as well as treatment that reduces or eliminates infection by a pathogen. Treatment includes any steps taken to prevent, cure, delay the onset of, reduce the severity of, or ameliorate one or more symptoms associated with an infection.

In some embodiments, a compound having the following structure (I) is provided:

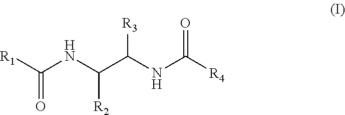

wherein:
R₁ is

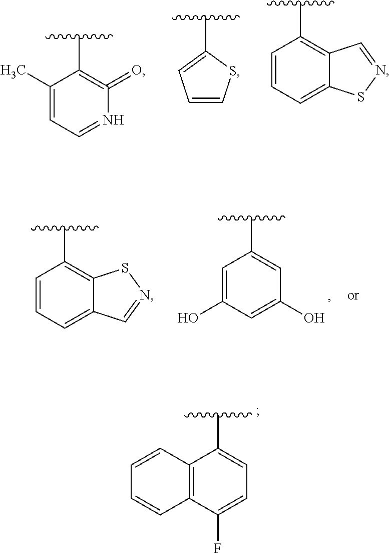

R₂ is H;
R₃ is

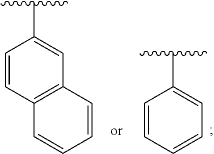 [SMILES: *c1ccc2ccccc2c1 ; *c1ccccc1]

and
R₄ is

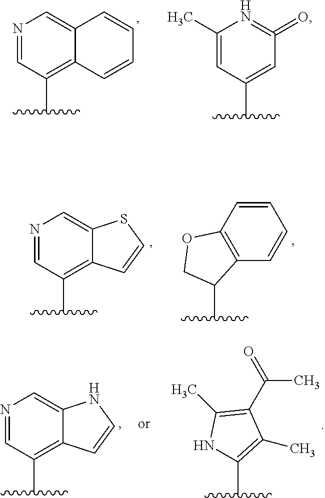

In some embodiments, a compound is provided that has one of the following structures:

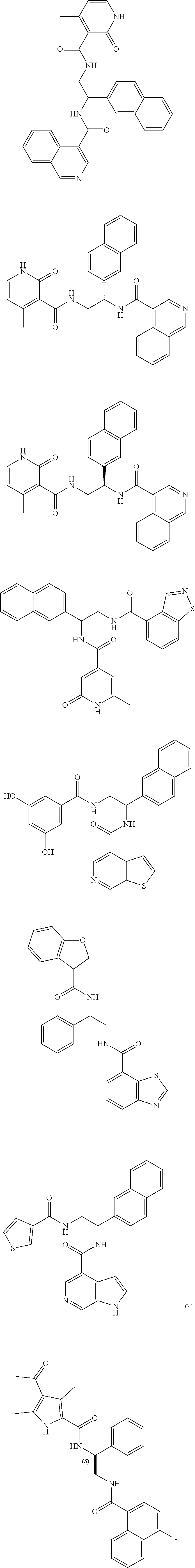

The compounds as described herein can be prepared according to any desired synthetic scheme. While exemplary synthetic schemes are provided herein with reference to the disclosed examples, in other embodiments, any desired synthetic route can be used to obtain the disclosed compounds. Further, in other embodiments, the disclosed compounds may exist as prodrugs, pharmaceutically acceptable salts, in racemic form or in enantiomeric form, or the like, and all such forms are encompassed within the scope of various embodiments of the disclosed compounds.

In some embodiments, the compounds disclosed herein are used to inhibit a protease, including the SARS-CoV-2 M^{pro} or the M^{pro} from another strain of coronavirus, for example M^{pro} from other beta-coronavirus strains including SARS-CoV-1, CoV MERS, MERS-CoV, HCoV-OC43, HCoV-HKU1, or the like, or M^{pro} from an alpha-coronavirus strain including HCoV-229E, HCoV-NL63, or the like. In some embodiments, the compounds disclosed herein are further used to inhibit cathepsin L. In some embodiments, the compounds disclosed herein are used as antiviral agents, e.g. to prevent or slow replication of a coronavirus including beta-coronavirus strains including SARS-CoV-2, SARS-CoV-1, CoV MERS, MERS-CoV, HCoV-OC43, HCoV-HKU1, or the like, or alpha-coronavirus strains including HCoV-229E, HCoV-NL63, or the like.

In some embodiments, the compounds disclosed herein are used to treat a coronavirus infection in a mammalian subject. The compounds can be formulated for administration to the mammalian subject in a suitable manner now known or later developed for delivering therapeutic agents, including by being formulated into a suitable pharmaceutical composition. The compound can be administered to the mammalian subject using any suitable dosage regime or dosage regimen.

In some embodiments, the compounds disclosed herein are administered alone to treat a coronavirus infection in a mammalian subject. In some embodiments, the compounds disclosed herein are administered together with one or more additional coronavirus therapeutics. In some embodiments, the one or more additional coronavirus therapeutics are antiviral agents, for example, remdesivir, molnupiravir, ritonavir, nirmatrelvir, ritonavir/nirmatrelvir (Paxlovid™) or the like; immune modulators, for example baricitinib or the like; or monoclonal antibody treatments for coronavirus, for example bebtelovimab, tixagevimab, cilgavimab, bamlanivimab, etesevimab, casirivimab, imdevimab, sotrovimab, or the like; or other anti-coronavirus therapeutics now known or later developed.

The co-administration of the compounds as disclosed herein together with one or more coronavirus therapeutics may include administering the combination of active agents together, whether at the same time and/or formulated together into a single pharmaceutical composition, or at different times, e.g. through separate modes of administration or through sequential administration (e.g. via the administration of a course of one of the active agents followed in time by the administration of a course of the second one of the active agents).

In some embodiments, tablets and dosage forms containing the compounds disclosed herein are provided. In some embodiments, the tablets or dosage forms contain a dose of the active ingredient(s) that is suitable for administration according to a pre-determined administration schedule, e.g. once daily, twice daily, three times daily or four times daily. The tablets and dosage forms can further contain suitable carriers or excipients for administration, including those suitable for administration in oral form, e.g. a tablet or syrup, although other modes of administration (e.g. any form of parenteral administration or the like) can be used if desired.

The selection of appropriate dosages for administration is within the expected ability of the person skilled in the art based upon the particular physical properties of the compound to be administered (e.g. rates of absorption, inactivation, excretion, the administration of any other active agents, and so on).

In various embodiments, the mammalian subject can be a human, monkey, cat, dog, sheep, rabbit, horse, cow, pig, goat, mouse, guinea pig, or the like. In some embodiments, the mammalian subject is a human.

In some embodiments, kits are provided, the kit containing a pharmaceutical formulation containing a compound as disclosed herein and instructions for the administration or use of the pharmaceutical formulation, for example according to one of the methods disclosed herein. In some embodiments, the kits are contained within suitable packaging.

### Examples

Further embodiments are further described with reference to the following examples, which are intended to be illustrative and not limiting in nature.

### Methods

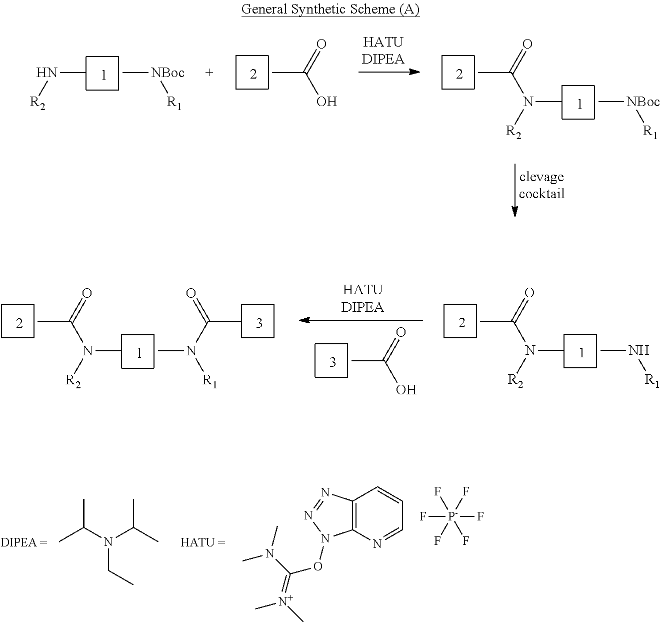

Compounds as described herein can be prepared according to any suitable synthetic scheme. For the examples described herein, the compounds were synthesized using General Synthetic Scheme (A). Briefly, Reagent (1) (1 eq.), Reagent (2) (1.1 eq.), HATU (Hexafluorophosphate Azabenzotriazole Tetramethyl Uronium) (1.1 eq.), and DIPEA (N,N-Diisopropylethylamine) (2.5 eq.) were mixed in DMSO (dimethyl sulfoxide) (approximately 0.7 ml per 100 mg of product). The mixture was sealed and stirred at ambient temperature for 16 hours. Then the cleavage cocktail (trifluoroacetic acid, triisopropylsilane, water (93:5:2; v/v) approximately 0.7 ml per 100 mg of product) was added to the mixture. The mixture was stirred at ambient temperature for 6 hours. The solvent was evaporated under reduced pressure, and to the residue DMSO (appr. 0.7 ml per 100 mg of product), DIPEA (5.7 eq.), Reagent (3) (1.2 eq.) and HATU (1.2 eq.) were added. The mixture was sealed and stirred at ambient temperature for 16 hours. The solution was filtered, analyzed by LCMS, and transferred for HPLC purification.

### Analytic Procedures

### LCMS Analysis

Mass spectrometry was carried out using an Agilent LC\MSD system with DAD\ELSD.

General Parameters: detection was carried out using the following parameters: DAD—DAD1A 215 nm, DAD1B 254 nm MSD—single quadrupole, AP-ESI (positive/negative mode switching); temperature was 25° C.

LCMS procedure 1: Column: InfinityLab Poroshell 120 SB-C18 4.6×30 mm 2.7 Micron with Guard: UHPLC Guard 3PK InfinityLab Poroshell 120 SB-C18 4.6×5 mm 2.7 Micron. Mobile phases: A—Deionized water: Formic acid (99.9:0.1%). B—HPLC-grade MeCN: (Deionized water: Formic acid (99.9:0.1%)) (95:5%). Gradient: from A—99%, B—1% to A—1%, B—99%.

### HPLC Procedure

Instrument specifications: Agilent 1260 Infinity systems equipped with DAD and mass-detector. General Parameters: temperature was 25° C.

HPLC procedure: Column: Chromatorex 18 SMB 100-5T 100A, 5 μm, 19 mm×100 mm with SiliaSphere C18 100A 5 μm 100 A, 19 mm×10 mm. Detection: DAD—DAD1A 215 nm, DAD1B 254 nm. MSD—single quadrupole, AP-ESI. Mobile phases: A—Deionized water (100%). B—HPLC-grade MeOH (100%).

### Chemical Synthesis and Results

Compound (101) (racemic mixture): N-{2-[(2-hydroxy-4-methylpyridin-3-yl)formamido]-1-(naphthalen-2-yl)ethyl}isoquinoline-4-carboxamide (101) was obtained by General Synthetic Scheme (A) using 57 mg (0.199 mmol) of tert-butyl N-[2-amino-2-(naphthalen-2-yl)ethyl]carbamate as Reagent (1), 36 mg (0.208 mmol) of isoquinoline-4-carboxylic acid as Reagent (2), 38.6 mg (0.252 mmol) of 2-hydroxy-4-methylpyridine-3-carboxylic acid as Reagent (3), 87.8 mg (0.231 mmol) of HATU, 67.8 mg (0.525 mmol) of DIPEA, 154.6 mg (1.196 mmol) of DIPEA, and 95.8 mg (0.252 mmol) of HATU. Product was purified by HPLC procedure (gradient: from A—30%: B—70% to A—0%: B—100%). Yield: 12.1 mg (12.1%). Beige powder. LCMS purity: 100% (LCMS procedure, R_{f}=0.55, run time=2 min). EI MS m/z: pos. 477.2 (MH⁺).

Separation of enantiomers (102) and (103): The enantiomers of N-{2-[(2-hydroxy-4-methylpyridin-3-yl)formamido]-1-(naphthalen-2-yl)ethyl}isoquinoline-4-carboxamide (101) were separated by HPLC on a Chiralpak IA column (250×4.6 mm, 5 mkm) using methanol as the mobile phase with a flow rate of 0.7 mL/min. The appropriate fractions were pooled and the solvent was evaporated to give each enantiomer. Compounds (102) and (103) are enantiomers.

(102): HPLC retention time=6.6 minutes, 100% enantiomeric purity by area under the curve. EI MS m/z: pos. 477.2 (MH⁺). 1H NMR (600 MHZ, DMSO-d6) δ 9.61 (m, 1H), 9.4 (s, 1H), 9.36 (m, 1H), 8.8 (s, 1H), 8.16 (m, 1H), 8.11-8.14 (m, 2H), 7.89-7.96 (m, 4H), 7.65-7.75 (m, 3H), 7.49-7.55 (m, 2H), 7.40 (m, 1H), 6.18 (d, 1H), 5.50 (m, 1H), 3.84 (m, 1H), 3.57 (m, 1H), 2.26 (s, 3H).

(103): HPLC retention time=16.7 minutes, 100% enantiomeric purity by area under the curve. EI MS m/z: pos. 477.2 (MH⁺). 1H NMR (600 MHZ, DMSO-d6) δ 9.61 (m, 1H), 9.4 (s, 1H), 9.36 (m, 1H), 8.8 (s, 1H), 8.16 (m, 1H), 8.11-8.14 (m, 2H), 7.89-7.96 (m, 4H), 7.65-7.75 (m, 3H), 7.49-7.55 (m, 2H), 7.40 (m, 1H), 6.18 (d, 1H), 5.50 (m, 1H), 3.84 (m, 1H), 3.57 (m, 1H), 2.26 (s, 3H).

Synthesis of (108): (S)-4-acetyl-N-(2-(4-fluoro-1-naphthamido)-1-phenylethyl)-3,5-dimethyl-1H-pyrrole-2-carboxamide (108) was obtained by General Synthetic Scheme (A) using 50 mg (0.212 mmol) of tert-butyl (S)-(2-amino-1-phenylethyl)carbamate as Reagent (1), 44 mg (0.231 mmol) of 4-fluoro-1-naphthoic acid as Reagent (2), 46.1 mg (0.254 mmol) of 4-acetyl-3,5-dimethyl-1H-pyrrole-2-carboxylic acid as Reagent (3), 88.7 mg (0.233 mmol) of HATU, 68.5 mg (0.530 mmol) of DIPEA, 126.1 mg (0.975 mmol) of DIPEA, and 96.8 mg (0.254 mmol) of HATU. Purified by HPLC procedure: (gradient: from A—45%: B—55% to A—0%: B—100%). Yield: 13 mg (13%). Powder. LCMS purity: 100% (LCMS procedure, R_{f}=0.61, run time=2 min). EI MS m/z: positive mode 472.2 (MH⁺).

### Synthesis of (104):

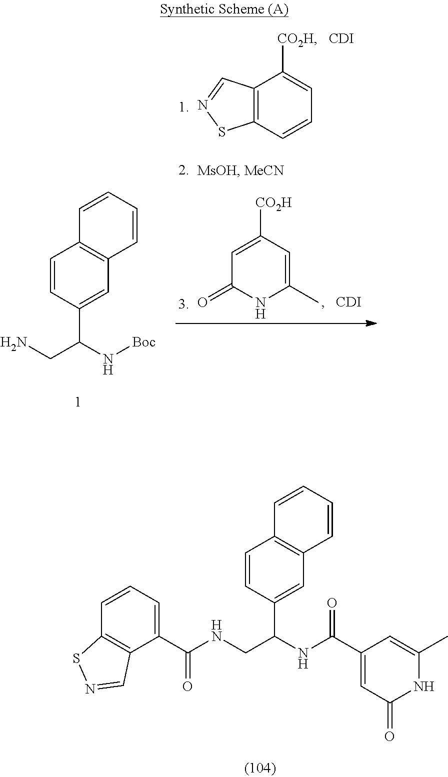

Compound (104) was synthesized following Synthetic Scheme (A). CDI (1,1′-Carbonyldiimidazole) (198 mg, 1.22 mmol) was added to a stirred solution of benzo[d]isothiazole-4-carboxylic acid (206 mg, 1.15 mmol) in acetonitrile (3 mL) at room temperature. The mixture was stirred at 50° C. for 30 min. Amine (1) (286 mg, 1 mmol) was added to the mixture and the reaction mixture was stirred at 50° C. for 1 h. The solvent was removed under reduced pressure and the residue was purified by preparative chromatography to give 273 mg (61%) of the intermediate. The intermediate compound was deprotected with MsOH (methanesulfonic acid) (288 mg, 3 mmol) in acetonitrile (3 mL) at room temperature.

CDI (1,1′-Carbonyldiimidazole) (198 mg, 1.22 mmol) was added to a stirred solution of 6-methyl-2-oxo-1,2-dihydropyridine-4-carboxylic acid (176 mg, 1.15 mmol) in acetonitrile (3 mL) and the mixture was stirred at 50° C. for 30 min. A solution of methanesulfonic acid salt from the previous step and N-methylmorpholine (0.6 mL, 5.45 mmol) were added to the mixture and the reaction mixture was stirred at room temperature overnight. The solution was concentrated under reduced pressure and the residue was dissolved in dichloromethane (10 mL). The solution was washed successively with water and a saturated aqueous solution of NaHCO₃ and dried over sodium sulfate. The solvent was removed under reduced pressure. The obtained crude product was purified by preparative chromatography to give 21 mg (7%) of the final product (104) as a pale yellow crystalline powder. ¹H NMR (400 MHZ, DMSO-d₆) δ 11.82 (s, 1H), 9.22 (s, 1H), 9.11 (d, J=8.4 Hz, 1H), 9.00 (t, J=5.9 Hz, 1H), 8.36 (dt, J=7.0, 1.3 Hz, 1H), 8.00-7.83 (m, 4H), 7.73-7.59 (m, 3H), 7.56-7.43 (m, 2H), 6.67 (s, 1H), 6.27 (s, 1H), 5.52 (q, J=7.6 Hz, 1H), 3.84 (t, J=6.7 Hz, 2H), 2.18 (s, 3H). LCMS (ESI, pos.): m/z=483.2 [M+H]+.

### Synthesis of (105):

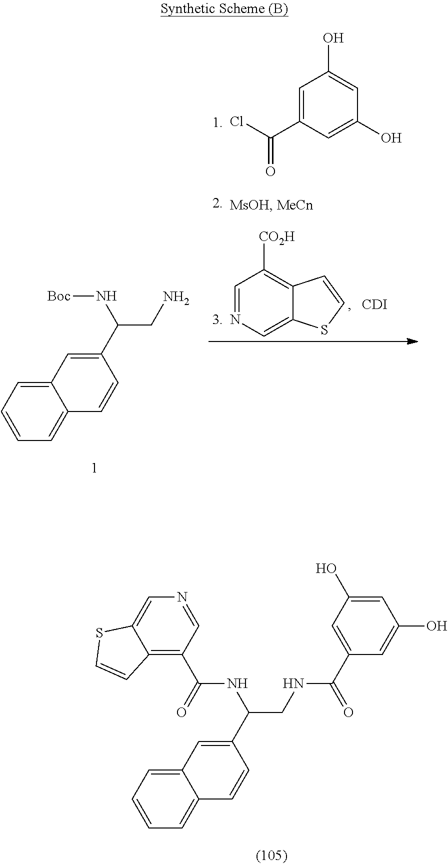

Compound (105) was synthesized following Synthetic Scheme (B). Briefly, 3,5-Dihydroxybenzoyl chloride (147 mg, 0.85 mmol) was added to a stirred solution of amine (1) (200 mg, 0.7 mmol) and N-methylmorpholine (0.2 mL, 1.8 mmol) in dichloromethane (5 mL) at 10° C. The reaction mixture was stirred at room temperature overnight. The mixture was washed with water and dried over sodium sulfate. The solvent was removed under reduced pressure. The residue was purified by preparative chromatography to give 83 mg (28%) of the intermediate compound. The compound was deprotected in a stirred solution with MsOH (methanesulfonic acid) (60 mg) in acetonitrile (3 mL) at room temperature overnight.

CDI (1,1′-Carbonyldiimidazole) (60 mg, 0.37 mmol) was added to a stirred solution of thieno[2,3-c]pyridine-4-carboxylic acid (60 mg, 0.33 mmol) in acetonitrile (2 mL). The mixture was stirred at 50° C. for 30 min. The solution of methanesulfonic acid salt from the previous step, N-methylmorpholine (0.1 ml, 0.91 mmol) and DMF (dimethylformamide) (10 mL) were added to the mixture. The reaction mixture was at room temperature overnight. The mixture was concentrated under reduced pressure. The residue was washed with water and a saturated aqueous solution of sodium bicarbonate and dried over sodium sulfate. The residue was dried in a vacuum and the obtained crude product was purified by preparative chromatography to give 47 mg (29%) of the final product (105) as a white solid. 1H NMR (400 MHZ, DMSO-d6) δ 9.44 (s, 2H), 9.40 (s, 1H), 9.24 (d, J=8.2 Hz, 1H), 8.91 (s, 1H), 8.52 (s, 1H), 8.16 (d, J=5.5 Hz, 1H), 7.96 (s, 1H), 7.93-7.88 (m, 3H), 7.79 (d, J=5.5 Hz, 1H), 7.65 (d, J=8.5 Hz, 1H), 7.55-7.42 (m, 2H), 6.69-6.55 (m, 2H), 6.32 (s, 1H), 5.60-5.46 (m, 1H), 3.89-3.61 (m, 2H). LCMS (ESI, pos.): m/z=484.0 [M+H]+.

### Synthesis of (106):

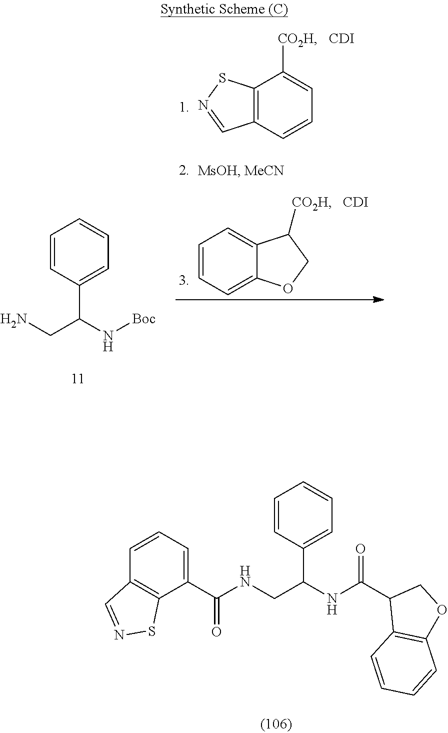

Compound (106) was synthesized following Synthetic Scheme (C). Briefly, CDI (1,1′-Carbonyldiimidazole) (198 mg, 1.22 mmol) was added to a stirred solution of benzo[d]isothiazole-7-carboxylic acid (206 mg, 1.15 mmol) in acetonitrile (3 mL) at room temperature. The mixture was stirred at 50° C. for 30 min. Amine (11) (1,1-Dimethylethyl N-(2-amino-1-phenylethyl)carbamate, CAS #142910-85-8) (236 mg, 1 mmol) was added to the mixture and the reaction mixture was stirred at 50° C. for 1 h. The solvent was removed under reduced pressure and the residue was purified by preparative chromatography to give 290 mg (73%) of the intermediate. The intermediate compound was deprotected with MsOH (methanesulfonic acid) (288 mg, 3 mmol) in acetonitrile (3 mL) at room temperature.

CDI (198 mg, 1.22 mmol) was added to a stirred solution of 2,3-dihydrobenzofuran-3-carboxylic acid (189 mg, 1.15 mmol) in acetonitrile (3 mL) and the mixture was stirred at 50° C. for 30 min. A solution of methanesulfonic acid salt from the previous step and N-methylmorpholine (0.6 mL, 5.45 mmol) were added to the mixture and the reaction mixture was stirred at room temperature overnight. The solution was concentrated under reduced pressure and the residue was dissolved in dichloromethane (10 mL). The solution was washed successively with water and a saturated aqueous solution of NaHCO₃ and dried over sodium sulfate. The solvent was removed under reduced pressure. The obtained crude product was washed with diethyl ether and purified by preparative chromatography to give 24 mg (7%) of the final product (106) as a white crystalline powder. 1H NMR (400 MHZ, DMSO-d6) δ 9.25-9.07 (m, 2H), 8.98-8.83 (m, 1H), 8.47-8.34 (m, 1H), 8.28-8.14 (m, 1H), 7.67 (t, J=7.4 Hz, 1H), 7.51-7.14 (m, 5H), 7.07 (t, J=7.6 Hz, 1H), 6.93-6.58 (m, 2H), 5.30-5.12 (m, 1H), 4.69-4.46 (m, 2H), 4.37-4.25 (m, 1H), 3.79-3.61 (m, 2H). LCMS (ESI, pos.): m/z=444.2 [M+H]+.

### Synthesis of (107):

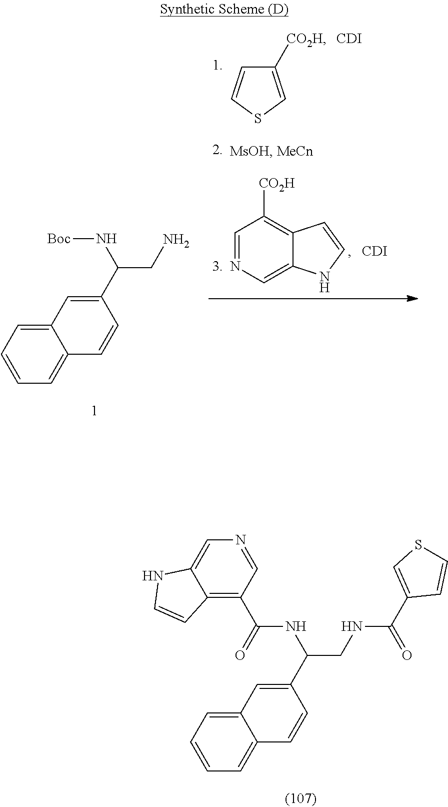

Compound (107) was synthesized following Synthetic Scheme (D). Briefly, CDI (1,1′-Carbonyldiimidazole) (198 mg, 1.22 mmol) was added to a stirred solution of thiophene-3-carboxylic acid (147 mg, 1.15 mmol) in acetonitrile (3 mL) at room temperature. The mixture was stirred at 50° C. for 30 min. Amine (1) (1,1-Dimethylethyl N-[2-amino-1-(2-naphthalenyl)ethyl]carbamate, CAS #946384-57-2) (286 mg, 1 mmol) was added to the mixture and the reaction mixture was stirred at 50° C. for 1 h. The solvent was removed under reduced pressure and the residue was purified by preparative chromatography to give 325 mg (82%) of the intermediate. The intermediate compound was deprotected with MsOH (methanesulfonic acid) (288 mg, 3 mmol) in acetonitrile (3 mL) at room temperature. CDI (198 mg, 1.22 mmol) was added to a stirred solution of 1H-pyrrolo[2,3-c]pyridine-4-carboxylic acid (186 mg, 1.15 mmol) in acetonitrile (3 mL) and the mixture was stirred at 50° C. for 30 min. A solution of methanesulfonic acid salt from the previous step and N-methylmorpholine (0.6 mL, 5.45 mmol) were added to the mixture and the reaction mixture was stirred at room temperature overnight. The solution was concentrated under reduced pressure and the residue was dissolved in dichloromethane (10 mL). The solution was washed successively with water (10 mL) and a saturated aqueous solution of NaHCO₃ (10 mL) and dried over sodium sulfate. The solvent was removed under reduced pressure. The obtained crude product was washed with diethyl ether and purified by preparative chromatography to give 27 mg (7%) of the final product (107) as an off-white powder. 1H NMR (400 MHZ, DMSO-d6) δ 11.82 (s, 1H), 9.03-8.96 (m, 1H), 8.85 (s, 1H), 8.64 (s, 1H), 8.58 (t, J=5.7 Hz, 1H), 8.07 (dd, J=3.0, 1.3 Hz, 1H), 7.95 (d, J=1.6 Hz, 1H), 7.93-7.86 (m, 3H), 7.70-7.64 (m, 2H), 7.55 (dd, J=5.1, 3.0 Hz, 1H), 7.52-7.46 (m, 2H), 7.44 (dd, J=5.0, 1.3 Hz, 1H), 6.81-6.78 (m, 1H), 5.52 (q, J=7.1 Hz, 1H), 3.78 (t, J=6.4 Hz, 2H). LCMS (ESI, pos.): m/z=441.2 [M+H]+.

### Enzyme Assays

Fluorogenic enzymatic assays to evaluate the inhibition of the SARS-CoV-2 M^{pro} protease by the tested compounds were carried out using the quenched fluorogenic substrate {DABCYL}-Lys-Thr-Ser-Ala-Val-Leu-GIn-Ser-Gly-Phe-Arg-Lys-Met-Glu-(EDANS)-NH2 (SEQ ID NO:1) as described in Tietjen et al., 2021. Briefly, the peptide substrate exhibits low fluorescence prior to cleavage, because the EDANS moiety is quenched by the DABCYL moiety. After the SARS-CoV-2 M^{pro} protease cleaves the substrate, the EDANS moiety is no longer quenched, resulting in an increase in fluorescence signal that is proportional to protease activity. Positive control, GC-376 (IC₅₀=31 nM), was tested in parallel with the synthesized compounds to ensure the accuracy of the assay.

Assays to evaluate the inhibition of cathepsin L contained 25 pM cathepsin L (RD systems: 952-CY-010), 5 uM Z-LR-AMC fluorogenic peptide substrate, 100 nL of test compound in 100% DMSO, in a total of 10 uL of 20 mM KPO₄, pH 6.0, 150 mM NaCl, 0.005% Tween20, 5 mM DTT in black low volume 384-well plates. The production of AMC (7-amino-4-methylcoumarin) was followed at 5 min intervals at 355 nm excitation, 460 nm emission in an Envision microplate reader (PerkinElmer). Reaction rates were determined by linear regression of the resulting progress curves. Rates were normalized to % inhibition, where 0% is equal to the rate in the presence of enzyme, and 100% is equal to the rate in the absence of enzyme. Nonlinear regression fits of the data to a one-site dose response curve were performed using XLFit (IDBS). Positive control, GC-376 (IC₅₀=2.0 nM), was tested in parallel with the test compounds to ensure the accuracy of the assay.

For select test compounds, the fluorogenic enzymatic assay measuring inhibition of SARS-CoV-2 M^{pro} was followed by a subsequent chromatographic purification by HPLC to remove any potential fluorescence interference from the test compound. Assays contained test compound, 20 nM M^{pro}, 10 uM substrate (DABCYL-KTSAVLQSGFRKME-EDANS, SEQ ID NO:1), 1 uL test compound in 100% DMSO, in a final volume of 100 uL of assay buffer (10 mM HEPES, pH 7.4, 150 mM NaCl, 5 mM DTT, 0.005% Tween20). After 1 hr, the reaction was stopped by the addition of 10 uL of 100 uM GC-376 (10 uM final concentration). Test compounds, substrate and product were then separated using an Agilent 1260 Infinity HPLC system with a Luna 5 um 50×4.6 mm C8 LC column (Phenomenex) using 10 mM sodium acetate, pH 4.0 as the aqueous phase and acetonitrile as the organic phase. Both absorbance at 420 nm and fluorescence at 355 nm excitation and 460 mm emission were followed. Product peak area was then normalized to % inhibition where 0% was equal to the peak area in the absence of test compound and 100% was equal to the peak area in the absence of enzyme, which was typically not detectable. Positive control, GC-376 (IC₅₀=10.7 nM), was tested in parallel with the test compounds to ensure the accuracy of the assay. As confirmed in this manner, compounds (101) and (106) had IC₅₀ values against the SARS-CoV-2 M^{pro} of 5.4 UM and 28 UM, respectively, confirming the accuracy of the fluorogenic assays for which the determined IC₅₀ values were 1.7 μM and 8.9 μM, respectively.

### Results

The results of the assays for each tested compound are summarized below in Table 1.

TABLE 1
Results of protease inhibition assays.

| | | | IC₅₀ | |
|---|---|---|---|---|
| | | | SARS- | |
| | | Mol. | CoV-2 | IC₅₀ |
| Compound | | Wt. | M^{pro} | Cathepsin L |
| # | Structure | (g/mol) | (μM) | (μM) |
| (101) | 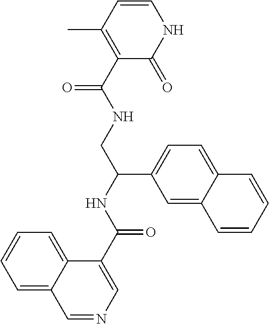 [SMILES: Cc1cc[nH]c(=O)c1C(=O)NCC(NC(=O)c1cncc2ccccc12)c1ccc2ccccc2c1] | 476.5 | 1.7 | > 100 |
| | [racemic mixture of enantiomers (102) | | | |
| | and (103)] | | | |
| (102) | 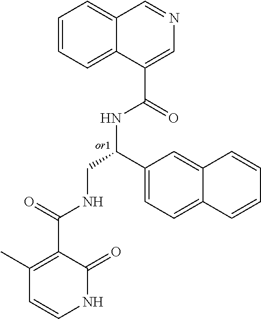 [SMILES: Cc1cc[nH]c(=O)c1C(=O)NC[C@H](NC(=O)c1cncc2ccccc12)c1ccc2ccccc2c1] | 476.5 | 1.6 | 47.8 |
| | wherein or1 is one of S and R, and the | | | |
| | opposite enantiomer of compound (103) | | | |
| (103) | 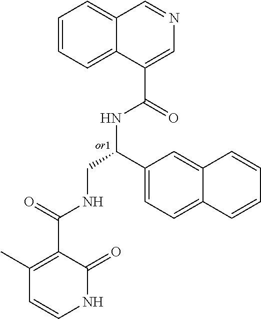 [SMILES: Cc1cc[nH]c(=O)c1C(=O)NC[C@H](NC(=O)c1cncc2ccccc12)c1ccc2ccccc2c1] | 476.5 | 71.2 | 36.8 |
| | wherein or1 is one of R and S, and the | | | |
| | opposite enantiomer of compound (102) | | | |
| (104) | 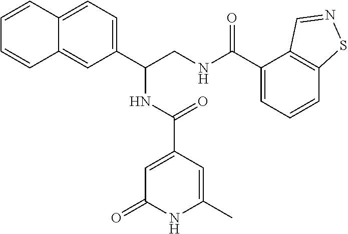 [SMILES: Cc1cc(C(=O)NC(CNC(=O)c2cccc3sncc23)c2ccc3ccccc3c2)cc(=O)[nH]1] | 482.6 | 47.8 | Not tested |
| (105) | 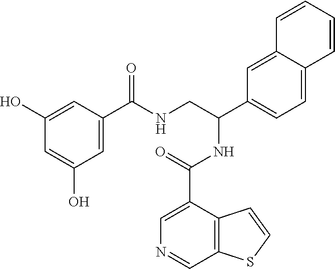 [SMILES: O=C(NCC(NC(=O)c1cncc2sccc12)c1ccc2ccccc2c1)c1cc(O)cc(O)c1] | 483.5 | 61.3 | Not tested |
| (106) | 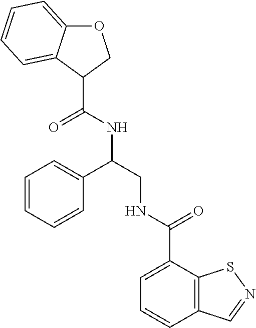 [SMILES: O=C(NCC(NC(=O)C1COc2ccccc21)c1ccccc1)c1cccc2cnsc12] | 443.5 | 8.9 | 10.2 |
| (107) | 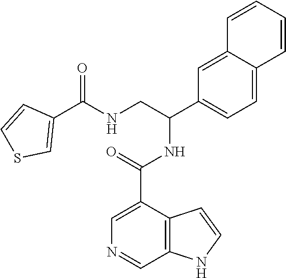 [SMILES: O=C(NCC(NC(=O)c1cncc2[nH]ccc12)c1ccc2ccccc2c1)c1ccsc1] | 440.5 | 19.2 | Not tested |
| (108) | 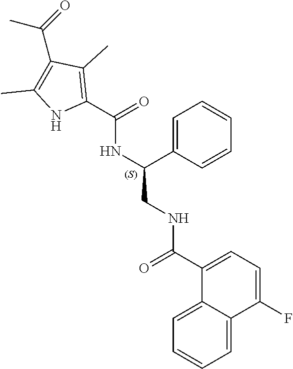 | 471.5 | 32.2 | Not tested |

### REFERENCES

The following references are of interest with respect to the subject matter disclosed herein. Each of the following references is incorporated by reference herein in its entirety.
Owen et al. “An oral SARS-CoV-2 M^{pro} inhibitor clinical candidate for the treatment of COVID-19.” Science, 374: 1586-1593, 2021.
Tietjen et al., “The Natural Stilbenoid (−)-Hopeaphenol Inhibits Cellular Entry of SARS-CoV-2 USA-WA1/2020, B.1.1.7, and B.1.351 Variants.” Antimicrob Agents Chemother 65(12):e00772-21, https://doi.org/10.1128/AAC.00772-21, 2021.
Unoh et al. “Discovery of S-217622, a non-covalent oral SARS-CoV-2 3CL protease inhibitor clinical candidate for treating COVID-19.” J. Med. Chem., 65: 6499-6512, 2022.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A compound having the following structure (I) or a pharmaceutically acceptable salt thereof: 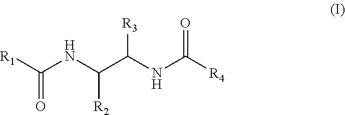
wherein:
R₁ is 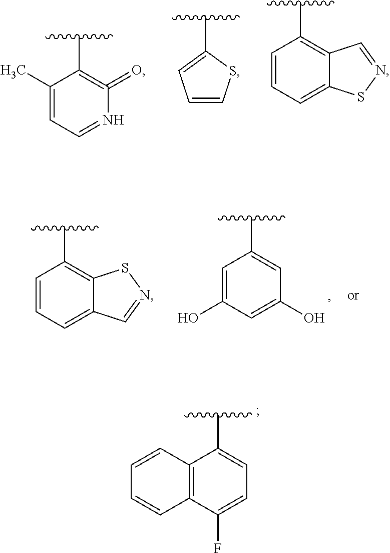
R₂ is H;
R₃ is 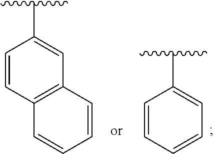 [SMILES: *c1ccc2ccccc2c1 ; *c1ccccc1]
and
R₄ is

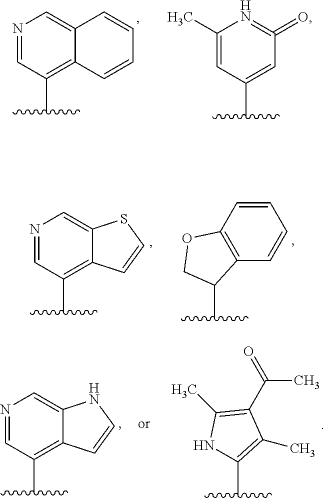

2. The compound as defined in claim 1, having one of the following structures or a pharmaceutically acceptable salt thereof:

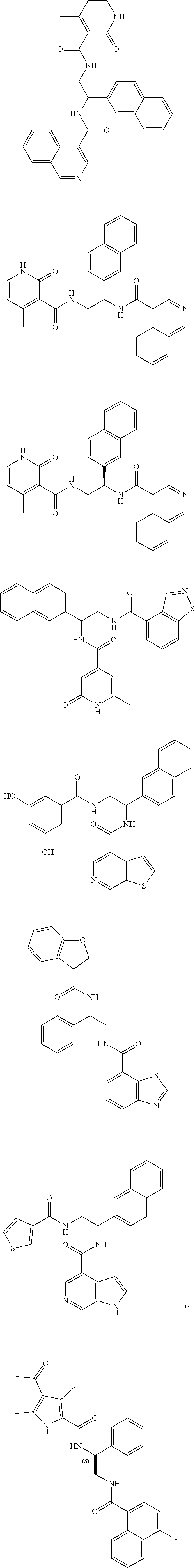

3. A pharmaceutical composition comprising a compound or a pharmaceutically acceptable salt as defined in claim 1 and a suitable excipient or carrier.

4. An oral dosage form comprising a pharmaceutical composition as defined in claim 3, optionally wherein the oral dosage form comprises a tablet.

5. A method of inhibiting a main protease (M^{pro}) of a coronavirus, the method comprising exposing the M^{pro} to the compound or pharmaceutically acceptable salt as defined in claim 1.

6. A method of inhibiting SARS-CoV-2 M^{pro}, the method comprising exposing the SARS-CoV-2 M^{pro} to the compound or pharmaceutically acceptable salt as defined in claim 1.

7. A method of inhibiting cathepsin L, the method comprising exposing the cathepsin L to the compound or pharmaceutically acceptable salt as defined in claim 1.

8. A method of inhibiting a main protease (M^{pro}) of a coronavirus in a mammalian subject, the method comprising administering the compound or pharmaceutically acceptable salt as defined in claim 1 to the mammalian subject.

9. A method of inhibiting SARS-CoV-2 M^{pro} protease in a mammalian subject, the method comprising administering the compound or pharmaceutically acceptable salt as defined in claim 1 to the mammalian subject.

10. The method as defined in claim 8, further comprising inhibiting cathepsin L in the mammalian subject.

11. A method of treating a coronavirus infection in a mammalian subject, the method comprising administering the compound or pharmaceutically acceptable salt as defined in claim 1 to the mammalian subject.

12. The method as defined in claim 11, wherein the coronavirus comprises a beta-coronavirus or an alpha-coronavirus.

13. The method as defined in claim 12, wherein the alpha-coronavirus comprises HCoV-229E or HCoV-NL63, or, wherein the beta-coronavirus comprises SARS-CoV, MERS-CoV, HCoV-OC43, or HCoV-HKU1.

14. The method as defined in claim 13, wherein the SARS-CoV comprises SARS-CoV-1 or SARS-CoV-2.

15. The method as defined in claim 14, wherein the SARS-CoV comprises SARS-CoV-2.

16. The method as defined in claim 11, the method further comprising administering a second coronavirus therapeutic to the mammalian subject, wherein the second coronavirus therapeutic is optionally administered at the same time as the compound, pharmaceutically acceptable salt, pharmaceutical composition or oral dosage form.

17. The method as defined in claim 16, wherein the second coronavirus therapeutic comprises a second antiviral, an immune modulator, or a monoclonal antibody treatment, wherein the second antiviral optionally comprises remdesivir, molnupiravir, ritonavir, nirmatrelvir or ritonavir/nirmatrelvir, the immune modulator comprises baricitinib, or the monoclonal antibody treatment comprises bebtelovimab, tixagevimab, cilgavimab, bamlanivimab, etesevimab, casirivimab, imdevimab, or sotrovimab.

18. The method as defined in claim 11, wherein the step of administering the compound or pharmaceutically acceptable salt to the mammalian subject comprises administering the compound or pharmaceutically acceptable salt orally or parenterally.

19. A kit comprising the compound or pharmaceutically acceptable salt as defined in claim 1 and instructions for carrying out a method of treating a mammalian subject for a coronavirus infection using the compound or pharmaceutically acceptable salt.

20. The method as defined in claim 11, wherein the mammalian subject is a human, monkey, cat, dog, sheep, rabbit, horse, cow, pig, goat, mouse, or guinea pig, optionally a human.


SEQUENCE LISTING (1 sequence)

SEQ ID NO: 1 - protein, 14 aa, synthetic construct
  MOD_RES 1: note = DABCYL
  MOD_RES 14: note = EDANS
KTSAVLQSGFRKME